(19) United States
(12) Patent Application Publication
(10) Pub. No.: US 2025/0325639 A1
(43) Pub. Date: Oct. 23, 2025

(54) ASSOCIATION OF A VEGETAL ACTIVE EXTRACT CONTAINING SUPEROXIDE DISMUTASE (SOD) AND A FENUGREEK EXTRACT AND USES THEREOF

(71) Applicants: BIONOV, EYRAGUES (FR); ROBERTET SA, GRASSE (FR)

(72) Inventors: Marion SABY, LE CRES (FR); Marina HUMBERT, GRASSE (FR); Laure EGOUMENIDES, MONTPELLIER (FR)

(21) Appl. No.: 18/566,310

(22) PCT Filed: Jun. 2, 2022
(86) PCT No.: PCT/EP2022/065120

§ 371 Date: Dec. 1, 2023

(30) Foreign Application Priority Data
Jun. 3, 2021 (EP) 21305747.4

(51) Int. Cl. A61K 38/44; A61K 36/42; A61K 36/48; A61P 25/28

(52) U.S. Cl. CPC A61K 38/446; A61K 36/42; A61K 36/48; A61P 25/28; A61K 2236/37; C12Y 115/01001

(57) ABSTRACT

The present invention concerns a product containing a vegetal active extract containing SOD and a fenugreek extract, and its uses for prevention or treatment of cognitive disorders, in particular in humans.

### FIELD OF THE INVENTION

The present invention relates to the field of treatment or prevention of cognitive disorders.

### BACKGROUND OF THE INVENTION

Optimal cognitive function mainly relies on the activity and communication between neurons, but can be impaired by numerous factors such as aging, cellular stress, chronic stress, and neurodegenerative disorders. Cognitive decline may be characterized by a decrease in performance in thinking, learning, memory, alertness, and/or impaired psychological skills, as well as by depression and anxiety. Psychobiological features of stress may also represent manifestations of oxidative stress, and long exposure to stress can induce or aggravate cognitive disorders. Normal aging also induces problems with memory, language, thinking or judgment.

So, it is essential to treat or prevent cognitive disorders for improving quality of life of human beings.

The Applicant specifically demonstrated that a combination of a vegetal active extract containing SODs, in particular a melon juice concentrate rich in superoxide-dismutase SOD, with fenugreek extract, has synergistic effects on reducing cognitive disorders.

It was previously known from Milesi et al. (2009) and confirmed by Carillon et al (2014) that oral supplementation with Extramel®, a coated freeze-dried melon juice concentrates rich in SOD (14 UI SOD/mg powder) sold by Bionov has a positive effect on several signs and symptoms of perceived stress, fatigue and quality of life (physical and mental health condition). But there is still a need to improve its performances on cognitive disorders and develop new products.

That was the goal answered by the present invention with combination of a melon juice extract rich in SOD with a fenugreek extract.

### SUMMARY OF THE INVENTION

A first object of the present invention is a product containing a vegetal active extract containing SOD and a fenugreek extract, in particular with at least one physiological acceptable excipient. In particular, the product contains a vegetal active extract of Cucumis melo rich in SOD, also named melon juice concentrate rich in SOD, and a fenugreek extract.

In a first embodiment, the product is a nutritional product, intended to be used in a healthy subject, meaning a subject exposed for example to stress, fatigue and/or aging, but not affected with pathological disorders.

In another embodiment, the product is a pharmaceutical product, intended to be used in a patient in need thereof, meaning non-healthy subject affected with pathological disorders.

The present invention also concerns the use of a nutritional product of the invention for improving cognitive function, in particular improving one condition selected from perception, memory, attention, and/or reasoning in a subject in need thereof.

Another object is the use of a nutritional product of the invention in the treatment or prevention of a condition selected from the group consisting of cognitive disorder, mood disorder, stress, and anxiety disorder.

Another object of the invention is the pharmaceutical product of the invention, for the treatment or prevention of neurodegenerative disease, in particular selected from the group consisting of Mild Cognitive Impairment (MCI), Alzheimer's disease, and Parkinson's disease in a patient in need thereof.

### Definitions

In a particular and preferred embodiment, the present invention uses a vegetable active extract containing SOD.

In plants, environmental adversity such as drought, high and low temperature, flood, presence of heavy metal and macronutrient deficit often leads to the increased generation of reduced oxygen species (ROS) and, consequently, SOD is suggested to play an important role in plant stress tolerance.

By vegetal active ‘extract’ or ‘concentrate’ rich in SOD, it means a raw or non-coated dried extract or concentrate containing a level of SOD of at least 60 IU/mg powder, in particular ranging from 80 to 180 IU/mg powder, preferably from 90 to 150 IU/mg powder measured according to the method of Zhou and Prognon (2006). In a particular embodiment, the concentrate rich in SOD contains a level of SOD of at least 90 IU/mg powder.

When the vegetal active ‘extract’ or ‘concentrate’ rich in SOD is in a coated form, it contains a level of SOD ranging from 5 to 25 SOD U/mg powder, in particular 10 to 20 SOD U/mg powder measured according to the method of Zhou and Prognon (2006), in particular 14 U SOD/mg powder.

As examples of vegetal active extract containing SOD, mention may be made of vegetal active extracts including but not limited to: Melon, Barley, Black plum, Cabbage, Cashew, Durum wheat, Grapevine, Maize, Papaya, Pea, Rice, Sugarcane, Tea plant, Wheat or Watermelon (Stephenie & al, 2015); but also marine plantae sources such as phytoplankton and algae, for example Prophyridium cruentum, Tetraselmis gracilis, Tetraselmis chuii, Bruguirea glymnorrhiza, Platymonas subcordiformis, Avicennia marina, Enteromorpha linza, or Sonneratia alba (Zeinaldi & al, 2020).

In a particular embodiment, the vegetal active extract containing SOD is selected from the group consisting of Melon, Maize, Papaya, Rice, Wheat, Watermelon and Phytoplankton.

In a preferred embodiment, the vegetal active extract containing SOD is a melon extract rich in SOD.

By “SOD” in the meaning of the present invention is an enzyme of superoxide dismutase type. It is to be noted that the superoxide dismutases of the invention are natural i.e. they are not chemically modified. In particular, the present invention concerns SODs in their entirety and not fragments thereof. SODs are classified into three categories according to the metal contained at their active site: manganese superoxide dismutases (Mn-SOD), copper and zinc superoxide dismutases (Cu/Zn-SOD) and iron superoxide dismutases (Fe-SOD).

As used herein, “cognitive function” refers to any mental process that involves symbolic operations, e.g perception, memory, attention, and reasoning. In one embodiment, “cognitive function” refers to memory.

As used herein, a “cognitive disorder” refers to any condition that impairs cognitive function. In one embodiment, “cognitive disorder” refers to learning disorder, attention deficit disorder (ADD), and attention deficit hyperactivity disorder (ADHD).

As used herein, a “stress-induced or stress-related cognitive dysfunction” refers to a disturbance in cognitive function that is induced, intensified or related to stress.

As used herein, a “mood disorder” refers to a disturbance in emotional state, such as depression, dysthymia, and bipolar disorder. In one embodiment, the mood disorder is depression.

As used herein, an “anxiety disorder” refers to a dysfunctional state of fear and anxiety, e.g., fear and anxiety that is out of proportion to a stressful situation or the anticipation of a stressful situation. In one embodiment, an anxiety disorder is any one or combination of generalized anxiety disorder, obsessive-compulsive disorder, panic disorder, post-traumatic stress disorder, and social anxiety disorder. In one embodiment, an anxiety disorder is a stress-induced anxiety disorder.

As used herein, “neurodegenerative disease” or, equivalently, “neurodegenerative disorder”, refers to any condition involving progressive loss of functional neurons in the central nervous system. In one embodiment, the neurodegenerative disease is associated with age-related cell death. Exemplary neurodegenerative diseases include, without limitation, Mild Cognitive Impairment (MCI), Alzheimer's disease, Parkinson's disease, Huntington's disease, amyotrophic lateral sclerosis (also known as ALS and as Lou Gehrig's disease), as well as AIDS dementia complex, adrenoleukodystrophy, Alexander disease, Alper's disease, ataxia telangiectasia, Batten disease, bovine spongiform encephalopathy (BSE), Canavan disease, corticobasal degeneration, Creutzfeldt-Jakob disease, dementia with Lewy bodies, fatal familial insomnia, frontotemporal lobar degeneration, Kennedy's disease, Krabbe disease, Lyme disease, Machado-Joseph disease, multiple sclerosis, multiple system atrophy, neuroacanthocytosis, Niemann-Pick disease, Pick's disease, primary lateral sclerosis, progressive supranuclear palsy, Refsum disease, Sandhoff disease, diffuse myelinoclastic sclerosis, spinocerebellar ataxia, subacute combined degeneration of spinal cord, tabes dorsalis, Tay-Sachs disease, toxic encephalopathy, and transmissible spongiform encephalopathy. In one embodiment, a neurodegenerative disease is selected from the group consisting of Mild Cognitive Impairment (MCI), Alzheimer's disease, amyotrophic lateral sclerosis, Huntington's disease, and Parkinson's disease. In one embodiment, a neurodegenerative disease is Alzheimer's disease. In another embodiment, a neurodegenerative disease is Mild Cognitive Impairment (MCI).

### BRIEF DESCRIPTION OF THE FIGURES

FIG. 1: Effects of combination of the invention (Cpd X) on novel object recognition (novel object session) at day 3. n is 10-12 per group. $$ p<0.01 $$$ p<0.001 vs theoretical value of 50% in discrimination index; Wilcoxon signed rank test (top/bottom-left). ***p<0.001 vs. Sc.Aβ/Veh group; #p<0.05, ##p<0.01 vs. Aβ25-35/Veh group; Dunnett's test after one-way ANOVA (top/bottom-right).

FIG. 2: Effects of combination of the invention (Cpd X) on lipid peroxidation in the hippocampus. n is 6 per group. ***p<0.001 vs. Sc.Aβ/Veh group; ###p<0.001 vs. Aβ25-35/Veh group; Dunnett's test after one-way ANOVA.

FIG. 3: Effects of combination of the invention (Cpd X) on amyloid-beta1-42 level in the hippocampus. n is 10-12 per group. ***p<0.001 vs. Sc.Aβ/Veh group; ###p<0.001 vs. Aβ25-35/Veh group; Dunnett's test after one-way ANOVA.

FIG. 4: Effects of combination of the invention (Cpd X) on pTau level in the hippocampus. n is 10-12 per group. ***p<0.001 vs. Sc.Aβ/Veh group; #p<0.05, ###p<0.001 vs. Aβ25-35/Veh group; Dunnett's test after one-way ANOVA.

FIG. 5: Effects of combination of the invention (Cpd X) on PSD-95 levels in the cortex. n is 10-12 per group. ***p<0.001 vs. Sc.Aβ/Veh group; ##p<0.01 vs. Aβ25-35/Veh group; Dunnett's test after one-way ANOVA.

FIG. 6: Effects of combination of the invention (Cpd X) on synaptophysin levels in the cortex. n is 10-12 per group. ***p<0.001 vs. Sc.Aβ/Veh group; ###p<0.001 vs. Aβ25-35/Veh group; Dunnett's test after one-way ANOVA.

FIG. 7: Effects of combination of the invention (Cpd X) on caspase-9 levels in the cortex. n is 10-12 per group. **p<0.01 vs. Sc.Aβ/Veh group; ###p<0.001 vs. Aβ25-35/Veh group; Dunnett's test after one-way ANOVA.

FIG. 8: HPLC-DAD(280 nm) profile of Fenugreek extract.

### DETAILED DESCRIPTION OF EMBODIMENTS OF THE INVENTION

The present invention concerns a product containing one or more superoxide dismutase(s) (SOD) and a fenugreek extract, in particular a vegetal active extract containing SOD and a fenugreek extract.

In the meaning of the present invention, the term ‘product’ encompasses a ‘composition’ containing SOD (preferably a vegetal active extract containing SOD) and fenugreek extract in a sole product, with at least one physiological acceptable excipient, and alternatively a kit wherein the SOD (preferably a vegetal active extract containing SOD) and the fenugreek extract are in separate compositions for simultaneous, sequential or delayed administration.

### SOD and Vegetal Active Extract Containing SOD

The products of the present invention comprise one or more SOD and a fenugreek extract, preferably a vegetal active extract containing SOD.

In an embodiment, the SOD is in the form of a mixture of SODs of plant origin, characterized in that it is essentially consisting of 3 superoxide dismutases: a manganese superoxide dismutase, a copper and zinc superoxide dismutase and an iron superoxide dismutase in at least two isoforms. Such mixture of SODs of plant origin for example is disclosed in the international publication WO 2016/128531.

In a particular and preferred embodiment as disclosed in detail hereunder, the SOD is in the form of a vegetal active extract containing SOD.

As non-limitative examples, the vegetal active extract containing SOD used in the present invention is selected from the group consisting of Melon, Barley, Black plum, Cabbage, Cashew, Durum wheat, Grapevine, Maize, Papaya, Pea, Rice, Sugarcane, Tea plant, Wheat, Watermelon, and marine plantae sources such as phytoplankton and algae, for example Prophyridium cruentum, Tetraselmis gracilis, Tetraselmis chuii, Bruguirea glymnorrhiza, Platymonas subcordiformis, Avicennia marina, Enteromorpha linza, or Sonneratia alba.

In a particular embodiment, the vegetal active extract containing SOD used in the present invention is selected from the group consisting of Melon, Maize, Papaya, Rice, Wheat, Watermelon and Phytoplankton.

In a preferred embodiment, the vegetal active extract containing SOD used in the present invention is a melon extract rich in SOD, in particular a melon juice concentrate containing SOD as the one sold by Bionov.

### Melon Juice Concentrate Containing Superoxide Dismutase (SOD)

In a particular embodiment, the extract containing SOD is a vegetal active extract of melon, also named melon juice concentrate.

In the following description, the terms ‘vegetal active extract of melon’ or ‘melon juice concentrate’ are used interchangeably.

### Melon Juice Concentrate (Non-Coated Form)

In a particular embodiment, as disclosed in the U.S. Pat. No. 5,616,323 patent, the vegetal active extract of melon used in the present invention is obtained from Cucumis melo and has a superoxide dismutase enzyme activity greater than 30 enzyme units per mg of soluble proteins, in particular greater than 50 enzyme units per mg, preferably greater than 60 units per mg of soluble proteins.

As a particular embodiment, the type of Cucumis melo from which the extract is obtained is in particular described in International Patent Application WO 92/02622.

These vegetal active extracts can be obtained by any process in the art which makes it possible to recover the soluble substance. Advantageously, these vegetal active extracts (also named melon juice concentrate) can be obtained by physical treatments including crushing the melon, recovery of the pulp, centrifugation, filtration and freeze-drying.

In a particular embodiment, the melon juice concentrate is obtained by grinding or pressing, in aqueous medium, a Cucumis melo at a pH of about 7.5, followed by the recovery of the supernatant, especially by centrifugation or filtration for possible subsequent purification. The pH for such a process is preferably 5 to 9, which makes it possible to remain within optimal physiological conditions (without denaturation of the SODs). Preferably, this is a centrifugation which makes it possible to discharge the membranous debris. Finally, the melon juice concentrate is submitted to a freeze-drying step.

The melon juice concentrate used in the present invention contains superoxide dismutase and other compounds including carotenoids, vitamins, and inorganic elements such as magnesium, copper and zinc.

The raw or non-coated dried extract or melon juice concentrate of the invention contains a level of SOD of at least 60 IU/mg powder, in particular ranging from 80 to 180 IU/mg powder, preferably from 90 to 150 IU/mg powder measured according to the method of Zhou and Prognon (2006). In a particular embodiment, the concentrate rich in SOD contains a level of SOD of at least 90 IU/mg powder.

In a particular example, the following table 1 illustrated the composition of a batch of raw or non-coated melon juice concentrate of the invention:

TABLE 1

| | Level (per g) | |
|---|---|---|
| Superoxide dismutase | >90,000 | IU |
| Carotenoids | 14 | μg |
| Alpha lipoic acid | <10 | μg |
| Coenzyme Q10 | <0.5 | μg |
| Vitamin A | <0.21 | μg |
| Vitamin E | 6.08 | μg |
| Vitamin C | 342 | μg |
| Copper | 33.9 | μg |
| Selenium | 0.075 | μg |
| Zinc | 49.2 | μg |
| Calcium | 1580 | μg |
| Iron | 79.4 | μg |
| Magnesium | 1480 | μg |
| Manganese | 6.6 | μg |
| Phosphorus | 1770 | μg |
| Potassium | 23.25 | mg |
| Sodium | 1650 | μg |
| Total mineral | 31.46 | mg |
| Total phenolics | 3.46 | mg GAE |

### Coated Melon Juice Concentrate

In a particular and preferred embodiment, the melon juice concentrate containing superoxide dismutase (SOD) is coated and/or microencapsulated to protect its active ingredients (SOD, vitamins . . . ), from aqueous medium, enzymes, pH and temperatures variations, in particular for use via oral route.

In a particular embodiment, the melon juice concentrate of the invention is coated or microencapsulated in a fat-soluble agent based on a fatty substance as disclosed in the US 2002/0182269. In particular, the fat-soluble agent is of plant origin. Advantageously, it is chosen from the group consisting of hydrogenated oils; palm oil or oil from the heart of palm fruit; hydrogenated seeds; stearates, in particular chosen from stearins, stearic acid and its derivatives; waxes, in particular vegetable waxes such as carnauba waxes; fatty acid monodiglycerides; saturated C₁₄-C₂₀ fatty acid triglycerides and mixtures thereof. In a particular embodiment, the fat-soluble agent is hydrogenated oil based on plant oil, in particular chosen from the group consisting of hydrogenated coconut oil, hydrogenated palm oil, hydrogenated soybean oil or hydrogenated rapeseed oil and mixtures thereof. In a particular embodiment, is the melon juice concentrate is coated with hydrogenated palm oil.

In another embodiment, the melon juice concentrate of the invention is coated or microencapsulated in shellac gum as disclosed in the publication WO2013/153220.

In another embodiment, the melon juice concentrate of the invention is encapsulated in biodegradable polymer microparticles, as disclosed in the international publication WO2006/030111. In particular, the biodegradable polymer microparticles comprise each successively in sequence from the core outwards: A) a cationic or anionic hydrophilic biodegradable polymer nucleus consisting of a carbohydrate or polyol or polyamine crosslinked matrix, said matrix being derived in the volume by cationic or anionic groups and said matrix incorporating an active plant extract containing superoxide dismutase; B) an outer anionic hydrophilic biodegradable polymer layer oppositely charged relative to the core A, said layer being associated by chemical interactions, advantageously ionic with the core A and surface molecules or polymers not being grafted by covalent bonds on the outer surface of said layer B.

In a particular embodiment, the vegetal active extract containing SOD used according to the invention is a proprietary coated freeze-dried melon juice concentrate obtained by physical treatment (crushing the melon, recovery of the pulp, centrifugation, filtration, freeze-drying, coating to protect the SOD activity from digestive enzymes) of a not genetically modified variety of melon which contains high levels of SOD and other antioxidants as illustrated in the above table 1.

In a preferred embodiment, the melon juice concentrate containing SOD is a coated freeze-dried melon juice concentrate containing a level of SOD ranging from 5 to 25 SOD U/mg powder, in particular 10 to 20 SOD U/mg powder measured according to the method of Zhou and Prognon (2006), in particular 14 U SOD/mg powder, and advantageously also carotenoids, vitamins and inorganic elements such as magnesium, copper and zinc.

In a particular embodiment, the freeze-dried melon juice concentrate is coated with palm oil.

As an illustrative example, one capsule used according to the invention for oral administration contains 10 mg of said freeze-dried melon juice concentrate (140 U of SOD). The capsule usually also contains active supplement such as starch or maltodextrin.

The examples further disclosed in the description illustrate the effect of the product Extramel® sold by Bionov, that is a freeze-dried melon juice concentrate coated with palm-oil and containing 14 U SOD/mg powder. Such product may be prepared as disclosed in the example 1 of the application published under number US2002/0182269.

### Fenugreek Extract

Fenugreek (Trigonella foenum-graecum) is an annual plant in the family Fabaceae, with leaves consisting of three small obovate to oblong leaflets. It is cultivated worldwide as a semiarid crop. Its seeds and leaves are common ingredients in Indian cuisine. Constituents of fenugreek seeds include flavonoids, alkaloids, coumarins, vitamins and saponins. Fenugreek has been extensively used as a flavour enhancer in several traditional cuisines. Additionally, the medicinal properties of fenugreek such as anticarcinogenic, antidiabetic, antioxidant, hypocholesterolemic, anti-inflammatory and immunological properties, make it an important compound to be used in food and pharmaceutical industries. Studies revealed that chronic ailments such as cancer, diabetes, heart diseases, infections, inflammations, and many other human diseases can be averted by using the natural sources of compounds with bioactivity such as phenolics, saponins, flavonoids, alkaloids, and other natural antioxidants. Extraction of bioactive compounds have been carried out via different methods of extraction known from the man skilled in the art such as maceration, ultrasound-assisted extraction, and microwave-assisted extraction.

In a particular embodiment, fenugreek extract is a fenugreek extract obtained by the following method:
a) The fenugreek seeds are extracted with supercritical CO₂ extraction, and further delipidated
b) The delipidated fenugreek seeds are further extracted by an alcoholic extraction.

The supercritical CO₂ extraction is used to obtain a high added value extract from fenugreek seed, deodorized and defatted extract. Such extraction also increases purity of the extract and delipidation prevents the risk of oxidative rancidity of the extract.

In a particular embodiment, the fenugreek extract is obtained by the following steps:
a) The fenugreek seeds are extracted with supercritical CO₂ extraction, and further delipidated
b) The delipidated fenugreek seeds are further extracted in ethanol 80%, followed by a filtration, a concentration under 20% dry matter, a pasteurization, and finally an atomization with 10% maltodextrin, for obtaining a dry extract of fenugreek.

The dry fenugreek extract according to the invention, is a powder having more than 95% of dry matter, a granulometry of less than 250 μm and characterized by the the presence of 4OH Isoleucin (1,5-3,5%) and Trigonellin (2-5%) measured by HPLC/HPTLC method.

### HPLC: High Performance Liquid Chromatography/HPTLC: High Performance Thin Layer Chromatography.

These measures are obtained according to the following conditions:
Analysis LC/MS (Liquid chromatography-mass spectrometry)
Column: Normal phase 250×4.6 mm, 5 μm
Solvent composition: Ethanol/water 0.1% formic acid
Flow: 1 ml/min

In a particular embodiment, the dry fenugreek extract also contains one or more compounds selected in the group consisting of homo-orientine, vitexine, diosgenin, sarsapogenins, néotogogenin, and isoflavones. Such compounds were detected by HPLC method.

In a particular and preferred embodiment, the fenugreek extract used according to the invention is a fenugreek extract having a HPLC-DAD (280 nm) profile as illustrated in the FIG. 8 wherein the main compounds are homo-orientine, vitexine, and trigonelline.

This profile was obtained using the following conditions:
Wavelength: 280 nm
Column: Agilent Poroshell 120S-C18, 4.6*150 mm, 2.7 μm
Solvent composition: Water 0.15% formic acid/Acetonitrile according to the following table 2.

TABLE 2

| Time | % Water/formic acid | % Acetonitrile |
|---|---|---|
| 0-5 min | 95 | 5 |
| 5-35 min | 40 | 60 |
| 35-50 min | 15 | 85 |
| 50-55 min | 5 | 95 |

Injection volume 2 μl
Temperature column: 30° C.
Flow: 1 ml/min.

So, in a particular and preferred embodiment, the fenugreek extract used in the invention contains 4OH isoleucine and trigonelline, and advantageously also homo-orientine and/or vitexine.

In a particular and preferred embodiment, the fenugreek extract is sold by ROBERTET.

### Product of the Invention Containing SOD and Fenugreek Extract

In the meaning of the present invention, the term ‘product’ encompasses a ‘composition’ containing SOD (preferably a vegetal active extract containing SOD) and fenugreek extract in a sole product, and alternatively a kit wherein the SOD (preferably a vegetal active extract containing SOD) and the fenugreek extract are in separate compositions for simultaneous, sequential or delayed administration. So according to the invention, SODs (preferably a vegetal active extract containing SOD) and fenugreek extract are in the same composition or in separate compositions of a kit.

In a particular embodiment, the composition or kit also contains zinc. The man skilled in the art will add zinc in concentrations following the usual recommendations of daily administration of minerals.

In a preferred embodiment, the product of the invention is a composition comprising a coated freeze-dried melon juice concentrate, a fenugreek extract and zinc.

In the meaning of the present invention, the term “nutritional composition” or ‘nutritional product’ particularly encompasses nutraceutical compositions (particularly food supplements for example in solid or liquid form), health-food compositions and beverages, particularly of dietary or nutritional nature such as beverages with antioxidant properties. Preferably, a nutritional composition comprises nutraceutical compositions and health beverages such as beverages with antioxidant properties.

In the present description, by physiologically acceptable excipient is meant a compound or combination of compounds included in a product which does not cause secondary reactions and for example allows facilitated administering of the active compound(s), increased lifetime thereof and/or efficacy in the body, increased solubility in solution or improved shelf life. These acceptable excipients are well known and can be adapted by persons skilled in the art to the type and mode of administration of the selected active compound(s).

In a particular embodiment, the product of the invention containing SOD (preferably a vegetal active extract containing SOD) and fenugreek extract is intended for administration via oral, nasal or parenteral route, preferable oral route.

When the product of the invention is intended for nutritional use, it is advantageously intended for administration via oral route. In a particular and preferred embodiment, the product is a nutritional product with a food-grade excipient, in particular in the form of a tablet, hard capsule, soft capsule, effervescent tablet, sachet or stick to be diluted, chewing gum, beverages, juices, yoghurt, confectionery, biscuit or bars.

When the product of the invention is intended for pharmaceutical use, it is intended for administration via oral, nasal or parenteral route, preferably oral route.

So, another object of the invention is a pharmaceutical product with a pharmaceutical excipient. For example, the pharmaceutical product of the invention is in the form of a tablet, hard capsule, soft capsule, effervescent tablet, sachet or stick to be diluted, syrup, elixir, herbal tea, chewing gum, spray, aerosol or solution for injection.

In one preferred embodiment, the product of the invention is intended for administration via oral route.

The daily dose and efficient amounts of each component in the product of the present invention will be adapted by the man skilled in the art, depending to the subject to be treated, and will be defined based on the weight of the subject to be treated.

In a particular embodiment, for a human subject of 70 kg, the daily dose for a nutritional use, would be:
70 á 560 IU SOD, preferably 140 to 560 IU SOD (corresponding to 5 mg to 40 mg of a melon juice concentrate having 141 U SOD/mg, preferably 10 mg to 40 mg of a melon juice concentrate having 141 U SOD/mg),
and 200 to 1600 mg of Fenugreek extract, preferably 400 mg Fenugreek extract.

So, in a particular embodiment, the product of the invention is a nutritional product intended for a daily administration, in particular in a dose equal or equivalent to 1 to 8 IU SOD and 2,8 to 22.8 mg Fenugreek extract per kilogram (kg) body weight.

In a particular embodiment, the product of the invention contains 140 UI SOD and 400 mg fenugreek extract, either in the same composition or in separate compositions of a kit.

In a particular embodiment, the product for oral administration contains 10 mg of a coated freeze-dried melon juice concentrate (14 U SOD/mg powder) and 400 mg fenugreek extract as disclosed above.

In a particular embodiment, the product of the invention contains 140 UI SOD and 400 mg fenugreek extract, and zinc in concentrations as usually used for daily administration of minerals, either in the same composition or in separate compositions of a kit.

### Uses and Methods of Treatment

The present invention also relates to the use of a product containing SOD (preferably a vegetal active extract containing SOD) and fenugreek extract as defined above, for improving cognitive function, in particular improving one condition selected from perception, memory, attention, and/or reasoning in a subject in need thereof. In a particular embodiment, the product of the invention is a nutritional product and is intended for improving cognitive function of healthy subject, meaning subject not affected by pathological disease.

In a particular embodiment, the product of the invention is used in the treatment or prevention of a condition selected from the group consisting of cognitive disorder, mood disorder, stress, and anxiety disorder.

In a particular embodiment, the use of the product of the invention, in particular containing coated freeze-dried melon juice extract rich in SOD and fenugreek extract, will improve one condition selected in the group consisting of perception, memory, attention, and reasoning, in particular at least memory.

In a particular embodiment, the use of the product of the invention, in particular containing coated freeze-dried melon juice extract rich in SOD and fenugreek extract, will prevent or treat a disorder selected in the group consisting of learning disorder, attention deficit disorder (ADD), and attention deficit hyperactivity disorder (ADHD).

In a particular embodiment, the use of the product of the invention, in particular containing coated freeze-dried melon juice extract rich in SOD and fenugreek extract, will prevent or treat a mood disorder.

In a particular embodiment, the use of the product of the invention, in particular containing coated freeze-dried melon juice extract rich in SOD and fenugreek extract, will prevent or treat an anxiety disorder.

As some of the cognitive dysfunction is induced, intensified or related to stress, the product of the invention is intended to prevent or treat subjects submitted to stress.

In a particular embodiment, the nutritional product is intended for a daily administration, in particular in a dose equal or equivalent 1 to 8 IU SOD and 2,8 to 22.8 mg fenugreek extract per kilogram (kg) body weight.

In another application of the invention, the product of the invention is a pharmaceutical product for the treatment or prevention of neurodegenerative disease, in particular selected from the group consisting of Mild Cognitive Impairment (MCI), Alzheimer's disease, and Parkinson's disease in a patient in need thereof.

Other examples of neurodegenerative diseases include, without limitation, amyotrophic lateral sclerosis (also known as ALS and as Lou Gehrig's disease), as well as AIDS dementia complex, adrenoleukodystrophy, Alexander disease, Alper's disease, ataxia telangiectasia, Batten disease, bovine spongiform encephalopathy (BSE), Canavan disease, corticobasal degeneration, Creutzfeldt-Jakob disease, dementia with Lewy bodies, fatal familial insomnia, frontotemporal lobar degeneration, Kennedy's disease, Krabbe disease, Lyme disease, Machado-Joseph disease, multiple sclerosis, multiple system atrophy, neuroacanthocytosis, Niemann-Pick disease, Pick's disease, primary lateral sclerosis, progressive supranuclear palsy, Refsum disease, Sandhoff disease, diffuse myelinoclastic sclerosis, spinocerebellar ataxia, subacute combined degeneration of spinal cord, tabes dorsalis, Tay-Sachs disease, toxic encephalopathy, and transmissible spongiform encephalopathy. In one embodiment, a neurodegenerative disease is selected from the group consisting of Alzheimer's disease, amyotrophic lateral sclerosis, Huntington's disease, and Parkinson's disease. In one embodiment, the neurodegenerative disease is Alzheimer's disease. In another preferred embodiment, the neurodegenerative disease is Mild Cognitive Impairment (MCI).

The man skilled in the art will adapt the doses of each component of the product, depending of the disease to be treated and the patient condition.

The invention also concerns a method of treatment of a subject in need thereof comprising administration of a product containing SOD (preferably a vegetal active extract containing SOD) and fenugreek extract as defined above.

In particular, the method of the invention is for the treatment or prevention of neurodegenerative disease, in particular selected from the group consisting of Mild Cognitive Impairment (MCI), Alzheimer's disease, and Parkinson's disease.

The invention will be now illustrated in the non-limitative examples.

### EXAMPLES

### Example 1: Materials

### 1-1 Melon Extract (Extramel®)

The melon extract (also named melon juice concentrate) used in the following examples is sold by the company BIONOV under the trademark Extramel®. This extract, after coating with hydrogenated palm oil, has an SOD activity of 14 units per mg of extract. The composition of Extramel® contains 20% by weight of the active plant extract and 80% by weight of the fat-soluble agent.

### 1-2 Fenugreek Extract

The fenugreek extract used in the following example is obtained by the following steps:
a) The fenugreek seeds are extracted with supercritical CO₂ extraction, and further delipidated,
b) The delipidated fenugreek seeds are further extracted in ethanol 80%, followed by a filtration, a concentration under 20% dry matter, a pasteurization, and finally an atomization with 10% maltodextrin, for obtaining a dry extract of fenugreek.

The dry extract of fenugreek used in the following examples, is a powder having more than 95% of dry matter, a granulometry of less than 250 μm and characterized by the presence of 4OH Isoleucine (1,5-3,5%) and Trigonelline (1,5-4,5%) measured by HPLC/HPTLC method.

These measures were obtained according to the following conditions:
Analysis LC/MS (Liquid chromatography-mass spectrometry)
Column: Normal phase 250×4.6 mm, 5 μm
Solvent composition: Ethanol/water 0.1% formic acid
Flow: 1 ml/min

The fenugreek extract used in the following examples is a fenugreek extract having a HPLC-DAD (280 nm) profile as illustrated in the FIG. 8 wherein the main compounds are homo-orientine, vitexine, and trigonelline.

This profile was obtained using the following conditions:
Wavelength: 280 nm
Column: Agilent Poroshell 120S-C18, 4.6*150 mm, 2.7 μm
Solvent composition: Water 0.15% formic acid/Acetonitrile according to the following table 3.

TABLE 3

| Time | % Water/formic acid | % Acetonitrile |
|---|---|---|
| 0-5 min | 95 | 5 |
| 5-35 min | 40 | 60 |
| 35-50 min | 15 | 85 |
| 50-55 min | 5 | 95 |

Injection volume 2 μl
Temperature column: 30° C.
Flow: 1 ml/min.

The fenugreek extract used in the following examples is sold by ROBERTET.

### 1-3 Combination of the Invention: Melon Juice Concentrate and Fenugreek Extract

The combination of the invention, also named ‘blend’ or ‘Cpd X’ in the following examples for mouse tests, comprises 95 mg/kg mouse with respectively 24.6 mg of melon extract (corresponding to 24,61 U SOD) as disclosed in example 1-1+70.3 mg fenugreek extract as disclosed in example 1-2/kg mouse.

Indeed, the daily dose for mouse is defined according to the daily dose for human which is:
1401 U melon extract Extramel® (10 mg of a product at 141 U/mg)
and 400 mg fenugreek extract

The Food and Drug Evaluation Administration (FDA) has established a guidance document to provide common conversion factors for deriving a human equivalent dose (HED), i.e. a dose in humans anticipated to provide the same degree of effect as that observed in animals at a given dose (U.S DHHS FDA Guidelines, 2005).

The term HDE is usually used to refer to the human equivalent dose of the NOAEL (No Observed Adverse Effect Levels). This conversion is based on the normalization of doses to body surface area. As the body surface area varies with weight, the conversion factors are dependent on the weight of the animals. However, analyses conducted have demonstrated that a standard factor provides a reasonable estimate of the HED over a broad range of human and animal weights.

So, taken into account the average weight of an adult (70 kg) and the conversion factor for a mouse (12,3 as established in the FDA guidelines mentioned above), the daily dose for a mouse corresponds to:
24,61 U/kg mouse of melon extract Extramel® (1401 U/70 kg×12,3).
70.3 mg/kg mouse of fenugreek extract (400 mg/70 kg×12,3)

The said combination of the invention has been used in the following example 2.

### Example 2: Analysis of the Protective Properties of the Combination of the Invention in the Aβ25-35 Mouse Model of Alzheimer's Disease

Alzheimer's disease (AD) is characterized by progressive accumulation of intracellular neurofibrillary tangles, extracellular parenchymal senile plaques, and cerebrovascular deposits composed mainly of amyloid-β (Aβ) peptides.

Aβ peptide is a proteolytic product derived, through sequential proteolysis, from amyloid precursor protein (APP), which occurs as a result of cleavage by β-secretase. Mutations at the cleavage sites in APP increase the production of Aβ oligomers. The progressive accumulation of Aβ in the form of senile plaques, which is one of the pathologic hallmarks of Alzheimer's disease (AD), has been recognized as one of the major causes of AD pathology (Kawahara and Kuroda, 2000) by triggering neurotoxicity, oxidative damage, synapses loss and inflammation (Cummings et al., 1998;). The most abundant Aβ peptide form found in AD brain senile plaques are the 40 and 42 amino acid forms (Sisodia et al., 1990).

Nevertheless, the number of senile plaques in a particular region of the AD brain correlates poorly with the local extent of neuron death or synaptic loss, or with cognitive impairment. Recent studies show a robust correlation between the soluble Aβ forms level and the extent of synaptic loss and severity of cognitive impairment (for review Sakono et al., 2010). Interestingly, the shorter amyloid-β₂₅₋₃₅ (Aβ₂₅₋₃₅) represents the biologically active region of Aβ because it is the shortest fragment able to aggregate in fibrillary β-sheet structures (Sato et al., 1995). Moreover, the monomeric form of Aβ₂₅₋₃₅ shows neurotoxic effects (Clementi et al., 2005).

### 2-1 First Study

The purpose of this first study was to determine whether the combination of the invention could alleviate the pathology induced in mice injected intracerebroventricularly (ICV) with amyloïd β₂₅₋₃₅ peptide (Aβ₂₅₋₃₅).

The combination efficacy was evaluated starting 7 days after the peptide injection, on:
novel object recognition (NOR) test
lipid peroxidation (LPO)
Aβ₁₋₄₂ and pTau (T181)

The combination and vehicle were administered once a day (o.d.) per os (PO) by gavage for 30 days. Administration of test combination and vehicle started two weeks before the Aβ₂₅₋₃₅ peptide injection (D14) and it lasted until day 17 (D17).

### 2-1-1 Animals and Treatment Groups

Thirty-four (34) male Swiss mice (30-35 g) were used.
Three (3) animal groups were constituted in the following manner.

TABLE 4
Treatment groups in this study

| | Session |
|---|---|
| Group treatments | n1 |
| 1. Sc.Aβ + Vehicle solution, PO, o.d. from D −14 to D 16 | 10 |
| 2. Aβ₂₅₋₃₅ + Vehicle solution, PO, o.d. from D −14 to D 16 | 12 |
| 3. Aβ₂₅₋₃₅ + Test combination (95 mg/kg), PO, o.d. from D− | 12 |
| Total | 34 |

Sc.Aβ= scrambled Aβ₂₅₋₃₅ (control peptide)

From two weeks before the Aβ₂₅₋₃₅ peptides injection (D-14) until D16 vehicle and test combination were administered PO by gavage o.d.
From fourteen days before the Aβ₂₅₋₃₅ peptides injection (D-14) until the day before the peptide injection (D-01) vehicle was administered to groups #1 and #2
From fourteen days before the Aβ₂₅₋₃₅ peptides injection (D-14) until the day before the peptide injection (D-01) test combination was administered to group #3
On D01, 1 h after the vehicle or test combination administration, Sc.Aβ or oligomeric Aβ₂₅₋₃₅ peptides was injected intracerebroventricularly (ICV)
From the day of Aβ₂₅₋₃₅ peptides injection (D01) until (D17) vehicle was administered to groups #1 and #2
From the day of Aβ₂₅₋₃₅ peptides injection (D01) until (D17) test combination was administered to group #3
From D08 to D16, the novel object recognition (NOR) on D09, D10 and D11.
On D17, all animals were sacrificed.

From all mice anesthetized with ketamine/xylazine blood from cardiac puncture (500-600 μl) was collected to prepare plasma. Blood (800 μl) was taken with a syringe (S-Monovette 1.2 ml Hep-Lithium, Sarstedt) and placed into an Eppendorf tube for plasma preparation. Two aliquots of plasma (200 μl) were collected.

Brain was carefully collected and hippocampi (right and left), cortices areas (right and left) were dissected out. Cortices areas will be used in the second study further disclosed.

Right hippocampus (n=6/group) was used to determine lipid peroxidation (LPO) levels using a colorimetric method.

Left hippocampus (all samples/group) was used to determine Aβ₁₋₄₂ and pTau (T181) levels by ELISAs

### 2-1-2 Drug Administration

Test combination was solubilized in its vehicle (sterile water), freshly prepared prior each administration. Test combination was kept at 4° C. before administration.

Vehicle (sterile water) and test combination were administered as following:
Route: per os
Method: by gavage, using a stainless-steel cannula and a disposable plastic 1 ml syringe to allow accurate dosing. The test combination and vehicle were administered in a volume calculated according to the individual body weight of each mouse at 5 ml/kg.
Frequency:
Once a day with vehicle (groups #1 and #2)
Once a day with test combination (group #3)
Treatment length
from D-14 to D17 included for all mice (31 days)

### Aβ25-35:

Denomination: amyloid-β protein (25-35) (Aβ₂₅₋₃₅) human, mouse, rat
CAS: 131602-53-4
Supplier: Genepep (France)
Reference: GPS 13387
Molecular Weight: 1060.28
Storage Temp: −20° C.
Appearance: white powder
Vehicle: sterile bidistilled water

### Sc.Aβ:

Denomination: scrambled amyloid-β protein (25-35) (Sc. Aβ), human, mouse, rat
CAS: NA
Supplier: Genepep (France)
Reference: GPS 19507
Molecular Weight: 1131.34
Storage Temp: −20° C.
Appearance: white powder
Vehicle: sterile bidistilled water

### 2-1-3 Amyloid Peptide Injection

Male Swiss mice are anesthetized 5 minutes with isoflurane 2.5%. After being restrained and their head immobilized, they are injected in the lateral right ventricle of the brain through a 26-gauge stainless steel needle, 3.5 mm long using a 10 pL Hamilton syringe. An injection volume of 3 pL is delivered gradually within 30 s and the needle left in place for an additional 30 s before being removed (Haley & McCormick, 1957). Animals are treated with Aβ₂₅₋₃₅ peptide (9 nmol/mouse) or Sc.Aβ peptide (9 nmol/mouse), in a final volume of 3 pL/mouse, according to the previously described method (Maurice et al., 1996,; Villard et al., 2009).

### 2-1-4 Animals

Male Swiss mice, weighing 30-35 g, were housed in groups with access to food and water ad libitum, except during behavioral experiments. They were kept in a temperature and humidity-controlled animal facility on a 12 h/12 h light/dark cycle (lights off at 07:00 μm). Mice were numbered by marking their tail with permanent markers. All animal procedures were conducted in strict adherence to the European Union directive of Sep. 22, 2010 (2010/63/UE).

### 2-1-5 Novel Object Recognition Test (NOR Test)

From the D09 to D11, all mice performed novel object test to measure recognition memory. The task procedure consists of three sessions.
Session 1 (D09): mice were placed individually in a squared open-field (50 cm×50 cm×50 cm high) made of white plexiglass and a floor equipped with infrared light emitting diodes. Mice were habituated to the open-field during a 10-min duration session and their locomotor activity captured through an IR-sensitive camera and analyzed using the Ethovision® software (Noldus). The activity was analyzed in terms of total distance traveled (m), locomotor speed (cm/s) and percentage of presence in the 25×25 cm central area defined by the software.
Session 2 (D10): 24 hours after session 1, two identical objects (50 ml plastic vials with caps) were placed at defined positions (at two opposite edges of the central area). Each mouse was placed in the open-field and the exploratory activity was recorded for 10-min. The activity was analyzed using the nose tracking protocol, in terms of number of contacts with objects and duration of contacts.
Session 3 (D11): 24 h after session 2, the object in position #2 was replaced by a novel one (a soft plastic chair feet protection) differing in color shape and texture from the familiar object. Each mouse was placed again in the open-field and the exploratory activity was recorded during a 10-min session. The activity was analyzed similarly according two parameters: the preferential exploration index and the discrimination index. The preference index was calculated as the ratio of the number (or duration) of contacts with the object in position #2 over the total number (or duration) of contacts with the two objects. Therefore, a preference index above 50% indicates a novel object preference, below 50% familiar object preference and 50% no preference (Wang et al., 2007). The discrimination index, DI, is represented as DI=(#2−#1)/(#1+#2), that is the time difference between number of contacts (or duration) spent exploring novel object (#2) and familiar one (#1) over the total number (duration) of contacts with the two objects. The result can vary between +1 (more time spent with the novel object) and −1 (more time spent with the familiar one) and 0 indicates a null preference (Silver et al. 2007).

Animals showing less than 10 contacts with objects during the session 2 or session 3 are discarded from the study. Usually, it represents 10-15% of the animals (high attrition test). The videotrack system can follow 4 animals simultaneously. Animals are tested according to their numeration. Between two sessions the open-field and objects are cleaned using water followed by 50% ethanol solution.

### Results:

During the first day (D09) and second day (D10), no differential effect of treatment was observed during the same object session procedure, in term of the frequency or the time interaction.

During the third day (D11), one of the two objects was replaced with a new one. Mice were free to explore both of them. The interest for the new one represented an intact recognition memory.

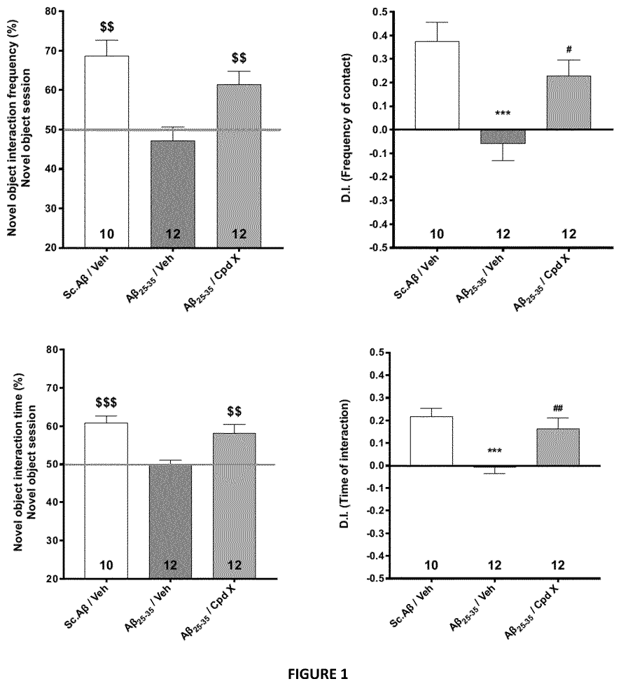

As illustrated in FIG. 1, Aβ₂₅₋₃₅ injection impaired highly significantly the recognition memory, as compared to Sc.Aβ mice (control). Combination of the invention (CpdX) very significantly and fully alleviated recognition memory deficits induced by Aβ₂₅₋₃₅ injection in terms of frequency of contacts (top left), or time of interaction (bottom left). In fact, values above 50% indicate a greater investigation of the novel object.

The same results were represented with the measure of discrimination index (DI): discrimination index of zero indicates the equal time spent around the two objects, whereas a positive value indicates more time investigating the new object as compared to the familiar one.

### 2-1-6 Euthanasia and Tissue Sampling

On D17, all animals were sacrificed.
All mice were anesthetized with ketamine/xylazine and blood from cardiac puncture (800 μl) was collected to prepare plasma. Blood was taken with a syringe (S-Monovette 1.2 ml Hep-Lithium, Sarstedt; Adaptateur multiple Luer Sartstedt) and placed into two Eppendorf tubes The tubes were inverted several times and kept at room temperature (RT) until plasma separation through centrifugation (2000 g for 10 min at 4 degrees Celsius). Two aliquots of plasma of 200 μl were prepared.
After that heart (left ventricle) was taken and collected into tubes and immediately frozen in liquid nitrogen.
Brain was collected quickly after decapitation and hippocampi (right and left into two Eppendorf tubes), cortices areas (right and left into two Eppendorf tubes) and rest of brain (in one Eppendorf tube) was weighed and frozen in liquid nitrogen.
Right hippocampus (n=6/group) was used to determine lipid peroxidation (LPO) levels using a colorimetric method.
Left hippocampus (all samples/group) was used to determine Ab1-42 and pTau(T181) levels by ELISAs.

### 2-1-7 Biochemical Analyses

### 2-1-7-1 Lipid Peroxidation Measurement by a Modified FOX Assay

Six (6) hippocampi from each group were used for LPO measurement as described by Hermes-Lima et al., 1995. After thawing, samples were homogenized in cold methanol (1/10 w/v), centrifuged at 1,000 g during 5 min and the supernatant placed in Eppendorf tubes. The reaction volume of each homogenate was added to FeSO₄ 1 mM, H₂SO₄ 0.25 M, xylenol orange 1 mM and incubated for 30 min at room temperature. After reading the absorbance at 580 nm (A₅₈₀1), 10 iil of cumene hydroperoxyde (CHP) 1 mM was added to the sample and incubated for 30 min at room temperature, to determine the maximal oxidation level. The absorbance is measured at 580 nm (A₅₈₀2).

The level of lipid peroxidation was determined as CHP equivalents according to: CHPE=A₅₈₀1/A₅₈₀2×[CHP (nmol)] and expressed as CHP equivalents per wet weight of tissue and as percentage of control group data (V-treated Sc.Aβ-administered mice).

### Results:

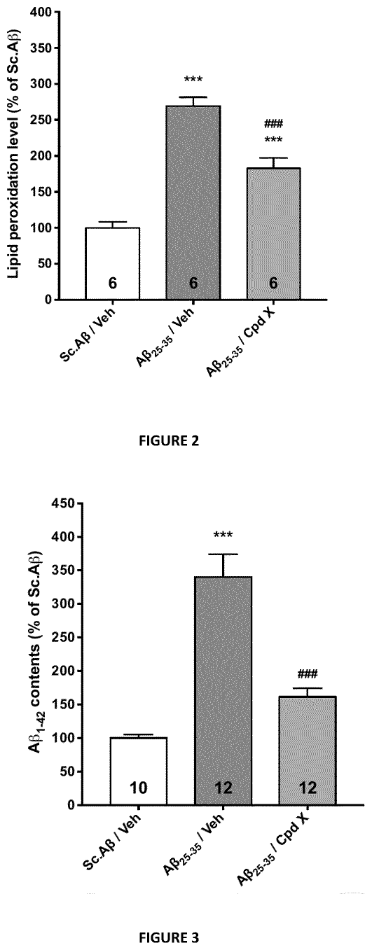

As illustrated in FIG. 2, Aβ₂₅₋₃₅ injection highly significantly increased the levels of LPO in the hippocampus, as compared to Sc.Aβ/V-treated mice.

Test combination (CpdX) very significantly but partially reduced the increase of oxidative stress induced by Aβ₂₅₋₃₅ injection.

### 2-1-7-2 Biomarkers Measurement

From n=6 samples/group, several markers were analyzed through ELISA method:
For Aβ₁₋₄₂ (left hippocampus), ELISA kit is from Cloud-Clone Corp.,
For pTau (T181), (left hippocampus), ELISA kit is from Fisher Scientific

After thawing, the hippocampus tissue (randomly chosen/blind selection) was homogenized in 50 mM Tris-150 mM NaCl buffer, pH 7.5, and sonicated for 20 s. After centrifugation (16,100 g for 15 min, 4° C.), supernatants were used for ELISA assays according to instructions of their respective manufacturer.

For all assays, absorbances were read at 450 nm and each samples concentration was calculated using the standard curve. All results are expressed in % of control Sc.Aβ treated group. All samples were assayed in duplicate and the average of these duplicates are used for calculi.

### Results

As illustrated in FIG. 3, Aβ₂₅₋₃₅ injection highly significantly increased the levels of Aβ₁₋₄₂ in the hippocampus, as compared to Sc.Aβ/N-treated mice. Test combination (CpdX), very significantly and fully inhibited the increase induced by Aβ₂₅₋₃₅ injection.

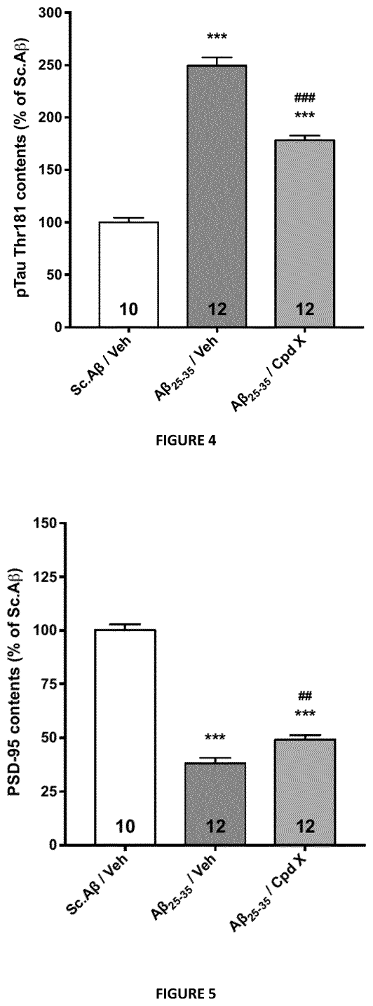

As illustrated in FIG. 4, Aβ₂₅₋₃₅ injection highly significantly increased the levels of pTau in the hippocampus, as compared to Sc.Aβ/N-treated mice.

Test combination (CpdX), very significantly but partially inhibited this increase induced by Aβ₂₅₋₃₅ injection.

### Statistical Analyses

All values, were expressed as mean±S.E.M. For the analysis of novel object recognition test, analyses were performed by one-way analysis of variance ANOVA (F value) followed by a Wilcoxon multiple comparison test. For the D.I., analyses were performed by one-way analysis of variance ANOVA (F value) followed by the Dunnett's post-hoc multiple comparison test.

Conclusion of this Study

All these results demonstrated that Aβ₂₅₋₃₅ injection highly significantly produced behavioral disturbance in terms of recognition memory, and also highly significantly increased the levels of oxidative stress (lipid peroxidation in hippocampus) and the Alzheimer disease hallmarks Aβ₁₋₄₂ & pTau in the hippocampus, as compared to Sc.A/V-treated mice.

Test combination (Cpd X), showed a beneficial preventive effect for the recognition memory in the novel object recognition paradigm, and also partially but significantly alleviated the increase of oxidative stress and hyperphosphorylation of Tau on Threonin 181, whereas it completely inhibited the increase of endogenous levels of Aβ₁₋₄₂ in the hippocampus.

### 2-2 Second Study

The purpose of the study was to determine whether test combination of the invention could alleviate the pathology induced in mice injected intracerebroventricularly (ICV) with amyloid-β₂₅₋₃₅ peptide (Aβ₂₅₋₃₅).

The treatment efficacy of test combination was evaluated ex vivo on the Aβ₂₅₋₃₅-induced modulation of post synaptic density 95 (PSD-95), synaptophysin (SYN) and caspase 9 expression in the cortex. PSD-95 (postsynaptic density protein 95) also known as SAP-90 (synapse-associated protein 90) and synaptophysin (SYN) are markers of synaptic alteration; caspase 9 is a marker of mitochondrial stress.

For this purpose, cortex samples taken from Aβ₂₅₋₃₅ injected mice of the first study were analyzed by ELISA.

### 2-2-1 ANIMALS AND TREATMENT GROUPS

Three (3) ELISA assays were performed from samples collected in first study disclosed above, to complete the biochemical changes determination:
PSD-95 levels in one cortex (right hemisphere)
Synaptophysin levels in one cortex (right hemisphere)
Caspase-9 levels in one cortex (right hemisphere)
   Samples were prepared from cortex of 10-12 mice/group (duplicate):

TABLE 5
Treatment groups in study

| | Session |
|---|---|
| Group treatments | n1 |
| 1. Cortex samples from Sc.Aβ + Vehicle solution, | 10 |
| PO, o.d. from D− | |
| 2. Cortex samples from Aβ₂₅₋₃₅ + Vehicle solution, | 12 |
| PO, o.d. from D− | |
| 3. Cortex samples from Aβ₂₅₋₃₅ + Test combination, | 12 |
| PO, o.d. from D− | |
| Total mice | 34 |

Sc.Aβ and Aβ₂₅₋₃₅ samples were homogenized in an extraction buffer specific to the commercial ELISA assay kit, as described by manufacturer and reference literature.
ELISA kits were:
   For PSD-95 ELISA kit is from Cloud-Clone Corp.,
   For synaptophysin ELISA kit is from Cloud-Clone Corp.,
   For caspase-9 ELISA kit is from Fine Test.

### 2-2-2 Biochemical Analyses by ELISA

n=10/12 cortex samples per group were used and several markers were analyzed through ELISA method according tu supplier's recommendations.

After thawing, the cortex tissue (randomly chosen/blind selection) was homogenized in 50 mM Tris-150 mM NaCl buffer, pH 7.5, and sonicated for 20 s. After centrifugation (16,100 g for 15 min, 4° C.), supernatants were used for the three ELISA assays according to instructions of their respective manufacturer.

For all assays, absorbances were read at 450 nm and each sample concentration was calculated using the standard curve. All results were expressed in % of control Sc.Aβ treated group. All samples were assayed in duplicate and the average of these duplicates were used for calculation.

### Statistical Analyses

All values were expressed as mean±S.E.M. Statistical analyses were performed separately for each compound using one-way ANOVA (F value), followed by the Dunnett's post-hoc multiple comparison test.

### Results:

As illustrated in FIG. 5, Aβ₂₅₋₃₅ injection highly significantly reduced the levels of PSD-95 in the cortex, as compared to Sc.Aβ/V-treated mice (control). Test combination, Cpd X, very significantly but partially inhibited this decrease.

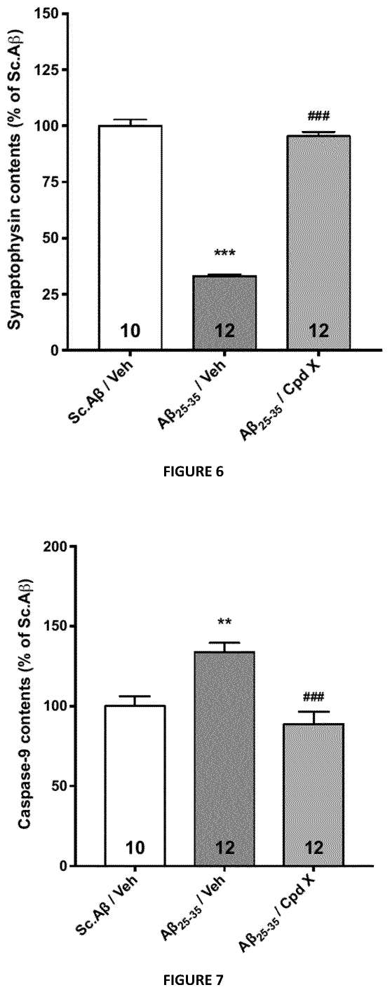

As illustrated in FIG. 6, Aβ₂₅₋₃₅ injection highly significantly reduced the levels of synaptophysin in the cortex, as compared to Sc.Aβ/V-treated mice (control). Test combination, Cpd X, very significantly and fully inhibited this decrease.

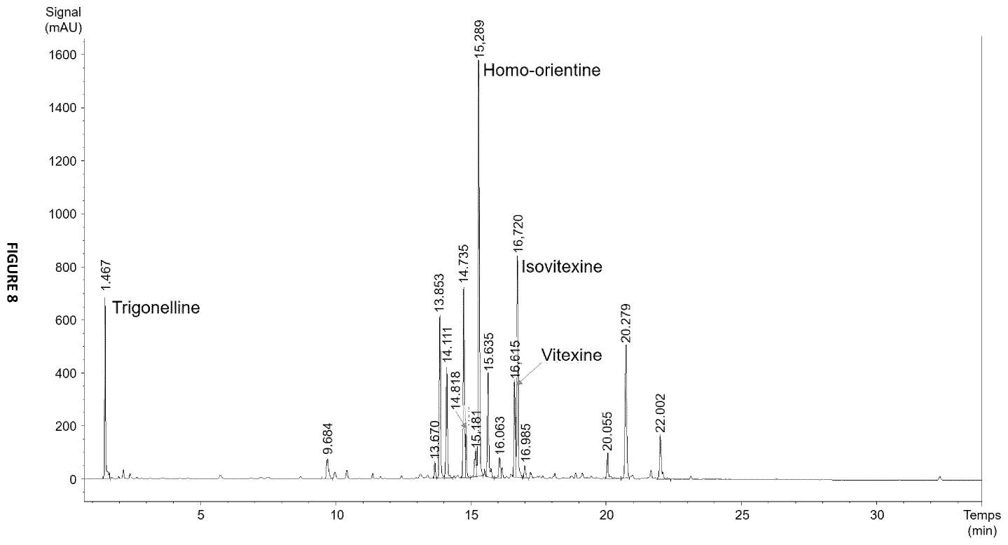

As illustrated in FIG. 7,

Aβ₂₅₋₃₅ injection highly significantly reduced the levels of Caspase-9 in the cortex, as compared to Sc.Aβ/V-treated mice (control). Test combination, Cpd X, very significantly and fully inhibited this decrease

So these results demonstrated that Aβ₂₅₋₃₅ injection highly significantly decreased the levels of the two synaptic markers PSD-95 and synaptophysin and very significantly increased the level of Caspase-9 in the cortex, as compared to Sc.Aβ/V-treated mice (control). Combination of the invention (Cpd X) partially but significantly inhibited the decrease of PSD-95 and fully inhibited the decrease of synaptophysin levels in the cortex.

Cpd X very significantly and fully inhibited the increase of caspase-9 levels in the cortex.

All the data resulted from these studies showed that the combination of the invention may be a relevant candidate to prevent or treat cognitive or neurological disorders, such as Alzheimer disease (AD).

### Example 3: Effects of Combination of the Invention Vs Each Component Alone, after 25-35 β-Amyloid Injury on Rat Primary Cortical Survival

The purpose of this study, in complement to previous studies disclosed above, was to determine the synergistic effect of the combination of the invention vs each component alone, on rat primary cortical neurons intoxicated with the human amyloid R₂₅₋₃₅ peptide, an in vitro Alzheimer's disease model.

The total number of cortical neurons, total length of neurite, caspase 3 expression and protein carbonylation were studied on neurons.

### 3-1 Cortical Neuron Cell Culture

Rat cortical neurons were cultured as described by Singer (1999). Briefly pregnant female rats of 15 days gestation were killed by cervical dislocation and the foetuses were removed from the uterus. The cortex was removed and placed in ice-cold medium of Leibovitz (L15; PanBiotech). Cortex was dissociated by trypsinisation (Trypsin EDTA 1×; PanBiotech). The reaction was stopped by the addition of Dulbecco's modified Eagle's medium (DMEM; PanBiotech) containing DNAase I grade II (0.1 mg/ml; PanBiotech) and 10% fetal calf serum (FCS; Invitrogen). Cells were then mechanically dissociated by 3 passages through a 10 ml pipette. Cells were then centrifuged at 515×g for 10 min at 4° C. The supernatant was discarded and the cell pellet was re-suspended in a defined culture medium consisting of Neurobasal (Invitrogen) supplemented with B27 (Invitrogen), L-glutamine (2 mM; PanBiotech), 2% PS (PanBiotech) and 10 ng/ml of BDNF (PeproTech). Viable cells were counted in a Neubauer cytometer using the trypan blue exclusion test. The cells were seeded at a density of 30 000 cells/well in 96 well-plates pre-coated with poly-D-lysine (Greiner) and were cultured at 37° C. in a humidified air (95%)/CO2 (5%) atmosphere.

### 3-2 β-Amyloid Preparation, Exposure and Drug Treatment: Protective Protocol

Amyloid-β₂₅₋₃₅ (Sigma) was reconstituted in define culture medium. After 10 days of culture, cells were pre-treated for 24 hours with test compounds or reference compound (Brain-Derived Neurotrophic Factor BDNF at 50 ng/mL) then intoxicated with Aβ₂₅₋₃₅.

The Aβ₂₅₋₃₅ preparation was used on primary cortical neurons after 11 days of culture at the final concentrations of 40 μM diluted in control medium for 48 hours incubation in order to induce a neuronal cell death of about 40%.
Active 1: fenugreek as disclosed in example 1.2
Active 2: melon extract Extramel® as disclosed in example 1.1
Blend (combination): active 1+active 2

The following conditions were done:

### Plate 1:

Control
Active 1 at 10 μg/mL; 3 μg/mL; 1 μg/mL; 0.3 μg/mL; 0.1 μg/mL+Aβ₂₅₋₃₅ (40 μM, 48 hours)
BDNF at 50 ng/ml+Aβ₂₅₋₃₅ (40 μM, 48 hours)
Control+Aβ₂₅₋₃₅ (40 μM, 48 hours)

### Plate 2:

Control
Active 2 at 35 μg/mL; 3.5 μg/mL; 1.05 μg/mL; 0.35 μg/mL; 0.105 μg/mL+Aβ₂₅₋₃₅ (40 μM, 48 hours)
BDNF at 50 ng/ml+Aβ₂₅₋₃₅ (40 μM, 48 hours)
Control+Aβ₂₅₋₃₅ (40 μM, 48 hours)

It should be noted that Active 2 did not dissolve in DMSO at a concentration of 10 mg/ml. Therefore, suspensions of the active ingredient at 35 μg/ml, 3.5 μg/ml, 1.05 μg/ml, 0.35 μg/ml and 0.105 μg/ml were prepared and the solutions were very strongly shaken before being incubated with the cells.

### Plate 3:

Control
Active 1 at 10 μg/mL+Active 2 at 3.5 μg/mL+Aβ₂₅₋₃₅ (40 μM, 48 hours)
Active 1 at 3 μg/mL+Active 2 at 1.05 μg/mL+Aβ₂₅₋₃₅ (40 μM, 48 hours)
Active 1 at 1 μg/mL+Active 2 at 0.35 μg/mL+Aβ₂₅₋₃₅ (40 μM, 48 hours)
Active 1 at 0.3 μg/mL+Active 2 at 0.105 μg/mL+Aβ₂₅₋₃₅ 5 (40 μM, 48 hours)
Active 1 at 0.1 μg/mL+Active 2 at 0.035 μg/mL+Aβ₂₅₋₃₅ (40 μM, 48 hours)
BDNF at 50 ng/ml+Aβ₂₅₋₃₅ (40 μM, 48 hours)
Control+Aβ₂₅₋₃₅ (40 μM, 48 hours)

One culture was performed with 6 wells per condition.

3-3 End Point Evaluation: Measurement of Total Number of Cortical Neurons and Neurite Length; Evaluation of the Caspase 3 and Protein Carbonyls-Stained Cortical Neurons after Aβ25-35 Intoxication and without Intoxication

After 48 hours of incubation, cells were fixed by a solution of 5% acetic acid (Sigma) and 95% ethanol (VWR) for 20 min at room temperature. The cells were then permeabilized with a solution of phosphate buffered saline (PBS; PanBiotech) with saponin (Sigma) and FCS for 15 min at room temperature. Cells were incubated with a mouse monoclonal anti-MAP-2 antibody (1/5 000, Sigma; ref: M4403) and with a rabbit monoclonal Anti-Caspase 3 antibody (1/500, Abcam; ref: ab13847) in PBS with saponin and FCS overnight at 4° C. These antibodies were revealed with an Alexa Fluor 488 goat anti-mouse IgG (1/400, Molecular probe, ref: A11001) and an Alexa Fluor 568 goat anti-rabbit IgG (1/400, Molecular probe, ref: A11011) in PBS with 1% FCS, 0.1% saponin, for 1 hour at room temperature. Nuclei of cells were labelled by a fluorescent marker (Hoechst solution, Sigma; ref: B11155) in the same solution. Cells were then labelled with an Alexa 647 carbonyls probe (1/1000, OXIProteomics) in PBS 1 h at room temperature.

For each well of culture, 20 pictures per well were taken using InCell AnalyzerTM 2200 (GE Healthcare) with 20× magnification. All the images were taken in the same conditions. The number of cortical neurons, the length of neurite, the level of caspase 3 expression and the level of protein carbonylation on neuronal cell body were automatically evaluated with Developer system analysis (GE Healthcare).

### Statistics

The data were expressed as mean±s.e.mean (of 6 data per condition, 1 culture). A global analysis of the data was performed using a one-way analysis of variance (ANOVA) following by Dunnett's test. The level of significance is set at p<0.05.

### Results

### Neurone Survival

TABLE 6
Neuron Survival

| | | | | | | 3 | |
|---|---|---|---|---|---|---|---|
| | | | | | | Active 1 | |
| | | | | 1 | 2 | (3 μg/ml) + | 4 |
| | Control | | | Active 1 | Active 2 | Active 2 | Active 1 |
| | medium | Aβ | BDNF + | (3 μg/ml) + | (1.05 μg/ml) + | (1.05 μg/ml) + | (1 μg/ml) + |
| | (0.1% | 25-53 | Aβ 25-53 | Aβ 25-53 | Aβ 25-53 | Aβ 25-53 | Aβ 25-53 |
| | DMSO | (40 μM) | (40 μM) | (40 μM) | (40 μM) | (40 μM) | (40 μM) |
| % of number | 100 | | | 86.04 | 44.45 | 85.93 | 55.42 |
| of cortical neuron | 100 | | | 61.31 | 68.65 | 78.26 | 50.66 |
| (MAP-2 staining) | 100 | | | 74.50 | 50.50 | 82.42 | 69.87 |
| | 100 | | | 77.29 | 54.28 | 83.28 | 74.56 |
| | 100 | | | 74.31 | 58.00 | 78.12 | 75.01 |
| | 100 | | | 74.69 | 66.79 | 71.60 | 62.14 |
| Mean | 100 | 54 | 86 | 74.69 | 57.11 | 79.93 | 64.61 |
| SEM | 5 | 2 | 3 | 3.24 | 3.83 | 2.07 | 4.16 |
| Pvalue | **** | | **** | ** | NS | ** | NS |
| vs Aβ 25-53 | | | | | | | |
| Pvalue | | | | NS | *** | | ** |
| vs Blend | | | | | | | |

| | | 6 | | | 9 |
|---|---|---|---|---|---|
| | | Active 1 | | | Active 1 |
| | 5 | (1 μg/ml) + | 7 | 8 | (0.3 μg/ml) + |
| | Active 2 | Active 2 | Active 1 | Active 2 | Actifve 2 |
| | (0.35 μg/ml) + | (0.35 μg/ml) + | (0.3 μg/ml) + | (0.105 μg/ml) + | (0.105 μg/ml) + |
| | Aβ 25-53 | Aβ 25-53 | Aβ 25-53 | Aβ 25-53 | Aβ 25-53 |
| | (40 μM) | (40 μM) | (40 μM) | (40 μM) | (40 μM) |
| % of number | 58.76 | 79.55 | 61.82 | 69.81 | 80.41 |
| of cortical neuron | 58.88 | 79.05 | 50.60 | 64.00 | 66.22 |
| (MAP-2 staining) | 57.07 | 83.13 | 63.40 | 66.38 | 61.92 |
| | 64.06 | 80.27 | 71.39 | 59.17 | 76.33 |
| | 61.67 | 77.40 | 63.66 | 65.22 | 68.08 |
| | 75.34 | 74.53 | 73.87 | 68.65 | 67.15 |
| Mean | 62.63 | 78.99 | 64.12 | 65.54 | 70.02 |
| SEM | 2.74 | 1.18 | 3.35 | 1.55 | 2.63 |
| Pvalue | NS | * | NS | * | NS |
| vs Aβ 25-53 | | | | | |
| Pvalue | *** | | NS | NS | |
| vs Blend | | | | | |

mean ± s.e.m;
*p < 0.05;
**p < 0.01;
***p > 0.001;
****p < 0.0001

The combination of the invention improves the neuronal survival. And the comparison of the result of condition 6 (fenugreek 1 μg/ml+Extramel®0,35 μg/ml), with conditions 1 (fenugreek 3 μg/ml) and 2 (Extramel®1,05 μg/ml), demonstrates a synergistic effect between the components of the combination, as the result is similar with three times less of each component.

TABLE 7
Neurite length

| | | | | | | 3 | |
|---|---|---|---|---|---|---|---|
| | | | | | | Active 1 | |
| | | | | 1 | 2 | (3 μg/ml) + | 4 |
| | | | | Active 1 | Active 2 | Active 2 | Active 1 |
| | Control | Aβ | BDNF + | (3 μg/ml) + | (1.05 μg/ml) + | (1.05 μg/ml) + | (1 μg/ml) + |
| | medium | 25-53 | Aβ 25-53 | Aβ 25-53 | Aβ 25-53 | Aβ 25-53 | Aβ 25-53 |
| | (0.1% DMSO | (40 μM) | (40 μM) | (40 μM) | (40 μM) | (40 μM) | (40 μM) |
| % of neurite | 100 | | | 70.7 | 37.3 | 101.6 | 42.0 |
| length | 100 | | | 42.7 | 58.0 | 86.8 | 41.1 |
| | 100 | | | 61.5 | 42.9 | 83.9 | 53.6 |
| | 100 | | | 62.4 | 40.9 | 86.1 | 59.1 |
| | 100 | | | 78.4 | 42.4 | 80.4 | 83.0 |
| | 100 | | | 87.6 | 59.5 | 87.1 | 52.6 |
| Mean | 100 | 42 | 75 | 67.2 | 46.8 | 87.6 | 51.9 |
| SEM | 7 | 4 | 5 | 6.36 | 3.85 | 2.97 | 3.62 |
| Pvalue | **** | | **** | ** | NS | **** | NS |
| vs Aβ 25-53 | | | | | | | |
| Pvalue | | | | * | **** | | **** |
| vs Blend | | | | | | | |

| | | 6 | | | 9 |
|---|---|---|---|---|---|
| | | Active 1 | | | Active 1 |
| | 5 | (1 μg/ml) + | 7 | 8 | (0.3 μg/ml) + |
| | Active 2 | Active 2 | Active 1 | Active 2 | Actifve 2 |
| | (0.35 μg/ml) + | (0.35 μg/ml) + | (0.3 μg/ml) + | (0.105 μg/ml) + | (0.105 μg/ml) + |
| | Aβ 25-53 | Aβ 25-53 | Aβ 25-53 | Aβ 25-53 | Aβ 25-53 |
| | (40 μM) | (40 μM) | (40 μM) | (40 μM) | (40 μM) |
| % of neurite | 48.0 | 88.3 | 47.0 | 64.1 | 77.5 |
| length | 50.6 | 89.4 | 38.4 | 61.1 | 77.7 |
| | 47.2 | 84.3 | 49.3 | 59.2 | 72.8 |
| | 50.1 | 82.4 | 55.8 | 46.1 | 81.8 |
| | 48.1 | 86.1 | 4 [symbol] .1 | 5 [symbol] .0 | 7 [symbol] .1 |
| | 77.9 | 82.3 | 63.5 | 80.6 | 74.7 |
| Mean | 53.6 | 85.5 | 50.0 | 61.2 | 77.1 |
| SEM | 4.88 | 1.22 | 3.53 | 4.63 | 1.26 |
| Pvalue | NS | **** | NS | NS | *** |
| vs Aβ 25-53 | | | | | |
| Pvalue | **** | | **** | ** | |
| vs Blend | | | | | |

mean ± s.e.m;
*p < 0.05;
**p < 0.01;
***p > 0.001;
****p < 0.0001 [symbol]
indicates data missing or illegible when filed

The combination of the invention improves the neurite length. And the comparison of the result of condition 12 (fenugreek 1 μg/ml+Extramel®0,35 μg/ml), with conditions 7 (fenugreek 3 μg/ml) and 8 (Extramel®1,05 μg/ml), demonstrates a synergistic effect between the components of the combination, as the result is similar with three times less of each component.

### Caspase 3 Expression

TABLE 8
Caspase 3

| | | | | | | 3 | |
|---|---|---|---|---|---|---|---|
| | | | | | | Active 1 | |
| | | | | 1 | 2 | (3 μg/ml) + | 4 |
| | | | | Active 1 | Active 2 | Active 2 | Active 1 |
| | Control | Aβ | BDNF + | (3 μg/ml) + | (1.05 μg/ml) + | (1.05 μg/ml) + | (1 μg/ml) + |
| | medium | 25-53 | Aβ 25-53 | Aβ 25-53 | Aβ 25-53 | Aβ 25-53 | Aβ 25-53 |
| | (0.1% DMSO | (40 μM) | (40 μM) | (40 μM) | (40 μM) | (40 μM) | (40 μM) |
| % of cortical neuron | | | | 1271.86 | 2027.57 | 436.25 | 1590.57 |
| (MAP-2 staining) [symbol] | | | | 724.89 | 1572.43 | 764.45 | 1319.65 |
| for the expression | | | | 656.73 | 1725.80 | 730.49 | 849.90 |
| of caspase [symbol] 3 | | | | 579.87 | 1651.18 | 804.79 | 971.73 |
| activated | | | | 588.04 | 1143.95 | 804.63 | 1075.52 |
| | | | | 440.04 | 1304.87 | 825.04 | 528.94 |
| Mean | 100 | 1643 | 497 | 710.24 | 1570.96 | 727.61 | 1056.05 |
| SEM | 9 | 134 | 90 | 118.80 | 128.07 | 59.90 | 150.89 |
| Pvalue | **** | | *** | ** | NS | **** | NS |
| vs Aβ 25-53 | | | | | | | |
| Pvalue | | | | NS | **** | | NS |
| vs Blend | | | | | | | |

| | | 6 | | | 9 |
|---|---|---|---|---|---|
| | | Active 1 | | | Active 1 |
| | 5 | (1 μg/ml) + | 7 | 8 | (0.3 μg/ml) + |
| | Active 2 | Active 2 | Active 1 | Active 2 | Actifve 2 |
| | (0.35 μg/ml) + | (0.35 μg/ml) + | (0.3 μg/ml) + | (0.105 μg/ml) + | (0.105 μg/ml) + |
| | Aβ 25-53 | Aβ 25-53 | Aβ 25-53 | Aβ 25-53 | Aβ 25-53 |
| | (40 μM) | (40 μM) | (40 μM) | (40 μM) | (40 μM) |
| % of cortical neuron | 1323.05 | 404.59 | 1697.67 | 1241.23 | 59 [symbol] .03 |
| (MAP-2 staining) [symbol] | 1984.65 | 536.49 | 981.75 | 1415.97 | 966.34 |
| for the expression | 1396.86 | 692.31 | 1195.77 | 969.74 | 923.38 |
| of caspase [symbol] 3 | 1321.92 | 1089.71 | [symbol] .09 | 769.95 | 888.01 |
| activated | 1638.03 | 1066.47 | 475.23 | 1389.45 | 1101.18 |
| | 1058.16 | 802.67 | 621.91 | 771.79 | 879.63 |
| Mean | 1453.81 | 765.37 | 964.74 | 1093.02 | 892.76 |
| SEM | 130.41 | 113.30 | 179.82 | 120.68 | 67.54 |
| Pvalue | * | NS | NS | **** | **** |
| vs Aβ 25-53 | | | | | |
| Pvalue | ** | | NS | NS | |
| vs Blend | | | | | |

mean ± s.e.m;
*p < 0.05;
**p < 0.01;
***p > 0.001;
****p < 0.0001 [symbol]
indicates data missing or illegible when filed

The combination of the invention inhibits the caspase 3 expression. And the comparison of the result of condition 21 (fenugreek 1 μg/ml+Extramel®0,35 μg/ml), with conditions 16 (fenugreek 3 μg/ml) and 17 (Extramel®1,05 μg/ml), demonstrates a synergistic effect between the components of the combination, as the result is similar with three times less of each component.

### Protein Carbonyls

TABLE 9
Protein carbonyls

| | | | | | | 3 | |
|---|---|---|---|---|---|---|---|
| | | | | | | Active 1 | |
| | | | | 1 | 2 | (3 μg/ml) + | 4 |
| | | | | Active 1 | Active 2 | Active 2 | Active 1 |
| | Control | Aβ | BDNF + | (3 μg/ml) + | (1.05 μg/ml) + | (1.05 μg/ml) + | (1 μg/ml) + |
| | medium | 25-53 | Aβ 25-53 | Aβ 25-53 | Aβ 25-53 | Aβ 25-53 | Aβ 25-53 |
| | (0.1% DMSO | (40 μM) | (40 μM) | (40 μM) | (40 μM) | (40 μM) | (40 μM) |
| % of cortical neuron | | | | 93.57 | 185.92 | 94.25 | 139.68 |
| (Map2 staining) | | | | 90.59 | 176.24 | 172.55 | 158.04 |
| positive for carbonyls | | | | 121.35 | 213.16 | 153.48 | 147.36 |
| | | | | 138.95 | 278.33 | 159.48 | 135.71 |
| | | | | 148.50 | 228.68 | 168.59 | 149.68 |
| | | | | 130.53 | 314.40 | 164.74 | 146.68 |
| Mean | 100 | 191 | 139 | 120.5 [symbol] | 232.79 | 152.18 | 146.19 |
| SEM | 10 | 11 | 12 | 9.74 | 22.01 | 11.91 | 3.20 |
| Pvalue | **** | | **** | **** | ** | **** | * |
| vs Aβ 25-53 | | | | | | | |
| Pvalue | | | | NS | ** | | NS |
| vs Blend | | | | | | | |

| | | 6 | | | 9 |
|---|---|---|---|---|---|
| | | Active 1 | | | Active 1 |
| | 5 | (1 μg/ml) + | 7 | 8 | (0.3 μg/ml) + |
| | Active 2 | Active 2 | Active 1 | Active 2 | Actifve 2 |
| | (0.35 μg/ml) + | (0.35 μg/ml) + | (0.3 μg/ml) + | (0.105 μg/ml) + | (0.105 μg/ml) + |
| | Aβ 25-53 | Aβ 25-53 | Aβ 25-53 | Aβ 25-53 | Aβ 25-53 |
| | (40 μM) | (40 μM) | (40 μM) | (40 μM) | (40 μM) |
| % of cortical neuron | 296.42 | 176.29 | 220.32 | 169.05 | 149.69 |
| (MAP-2 staining) | 237.89 | 131.12 | 183.93 | 198.00 | 207.01 |
| positive for carbonyls | 279.19 | 153.27 | 147.94 | 197.59 | 227.08 |
| | 246.12 | 150.65 | 166.77 | 158.65 | 218.30 |
| | 249.11 | 134.63 | 151.30 | 169.60 | 225.62 |
| | 270.56 | 174.20 | 160.79 | 226.24 | 231.24 |
| Mean | 263.21 | 153.36 | 171.84 | 186.52 | 209.82 |
| SEM | 9.21 | 7.77 | 11.01 | 10.33 | 12.52 |
| Pvalue | NS | **** | NS | **** | NS |
| vs Aβ 25-53 | | | | | |
| Pvalue | **** | | * | NS | |
| vs Blend | | | | | |

mean ± s.e.m;
*p < 0.05;
**p < 0.01;
****p < 0.0001 [symbol]
indicates data missing or illegible when filed

The combination of the invention inhibits the carbonylation of proteins. And the comparison of the result of condition 30 (fenugreek 1 μg/ml+Extramel® 0,35 μg/ml), with conditions 25 (fenugreek 3 μg/ml) and 26 (Extramel®1,05 μg/ml), demonstrates a synergistic effect between the components of the combination, as the result is similar with three times less of each component.

All these results demonstrate that the combination of the invention has a synergistic protective effect on neurodegenerative disease such as Alzheimer disease.

### Example 4: Formulations

The following formulations are based on an efficient amount of coated freeze-dried melon juice concentrate rich in SOD (14 U/mg powder) and Fenugreek extract for a human adult of 70 kg, but may be adapted to the weight of the subject to be treated.

### 4-1 Capsule 2 in 1

| Compounds | Weight (mg) |
|---|---|
| Coated freeze-dried melon juice | 5 mg |
| concentrate rich in SOD (14 UI/mg powder)* | (70 IU SOD) |
| Fenugreek extract* | 200 mg |
| Maltodextrine | 95 mg |

*as prepared in 1.1 and 1.2

The capsule “2 in 1” comprising the combination of the invention is prepared according to the classical method of formulation in capsule.

For a daily dose of 1401 U SOD, an oral administration of 2 capsules a day is adapted to provide the searched effects on cognitive and neurological disorders.

4-2 Kit with 1 Capsule Melon Juice Concentrate (100 mg) and 1 Capsule Fenugreek (500 mg)

As an alternative to the capsule “2 in 1”, the combination of the invention is formulated into a kit comprising 1 capsule melon and 1 capsule fenugreek.

Capsule melon juice concentrate rich in SOD

| Compounds | Weight (mg) |
|---|---|
| Coated freeze-dried melon juice | 10 mg |
| concentrate rich in SOD (14 UI/mg powder)* | (140 IU SOD) |
| Maltodextrine | 90 mg |

*as prepared in 1.1

Capsule fenugreek

| Compounds | Weight (mg) |
|---|---|
| Fenugreek extract* | 400 mg |
| Maltodextrine | 100 mg |

*as prepared in 1.2

### REFERENCES

Carillon et al. Dietary Supplementation with a Superoxide Dismutase-Melon Concentrate Reduce Stress, Physical and Mental Fatigue in Healthy People: A Randomised, Double-Blind, Placebo-Controlled Trial. Nutrients 2014, 6, 2348-2359; doi:10.3390/nu6062348.
Clementi M E, Marini S, Coletta M, Orsini F, Giardina B, Misiti F. AF3(31-35) and AF3(25-35) fragments of amyloid beta-protein induce cellular death through apoptotic signals: Role of the redox state of methionine-35. FEBS Letters. (2005); 579(13):2913-2918.
Cummings J L, Vinters H V, Cole G M, Khachaturian Z S. Alzheimer's disease: etiologies, pathophysiology, cognitive reserve, and treatment opportunities. Neurology. (1998); 51(1 Suppl 1):S2-17; discussion S65-7.
Hermes-Lima, M., Willmore, W. G., Storey, K. B. Quantification of lipid peroxidation in tissue extracts based on Fe(Ill)xylenol orange complex formation. Free Radic Biol Med, 19, 271-280, 1995.
Kawahara M, Kuroda Y. Molecular mechanism of neurodegeneration induced by Alzheimer's beta-amyloid protein: channel formation and disruption of calcium homeostasis. Brain Res Bull. (2000); 53(4):389-97.
Maurice T, Lockhart B P, Privat A. Amnesia induced in mice by centrally administered β-amyloid peptides involves cholinergic dysfunction. Brain Res 706, 181-193, 1996.
Milesi et al. Effect of an oral supplementation with a proprietary melon juice concentrate (Extramel®) on stress and fatigue in healthy people: a pilot, double-blind, placebo-controlled clinical trial. Nutrition Journal 2009, 8:40.
Sakono M, Zako T. 2010. Amyloid oligomers: formation and toxicity of Abeta oligomers. FEBS J 277:1348-58. Singer C, Figueroa-Masot X, Batchelor R, Dorsa D. Mitogen-activatedprotein kinase pathway mediates estrogen neuroprotection afterglutamate toxicity in primary cortical neurons. J Neurosci (1999); 19:2455-2463.
Sato K, Wakamiya A, Maeda T, Noguchi K, Takashima A, Imahori K. Correlation among secondary structure, amyloid precursor protein accumulation, and neurotoxicity of amyloid beta(25-35) peptide as analyzed by single alanine substitution. J. Biochem. (1995); 118(6):1108-1111.
Sisodia S S, Koo E H, Beyreuther K, Unterbeck A, Price D L. Evidence that beta-amyloid protein in Alzheimer's disease is not derived by normal processing. Science. (1990); 248(4954):492-5.
Stephenie S. et al. An insight on superoxide dismutase (SOD) from plants for mammalian health enhancement. Journal of Functional Foods 68 (2020) 103917.
Villard V, Espallergues J, Keller E, Alkam T, Nitta A, Yamada K, Nabeshima T, Vamvakides A, Maurice T. Anti-amnesic and neuroprotective effects of the aminotetrahydrofuran derivative ANAVEX1-41 against amyloid β25-35-induced toxicity in mice. Neuropsychopharmacology, 34, 1552-66, 2009.
Wang D, Noda Y, Zhou Y, Mouri A, Mizoguchi H, Nitta A, Chen W, Nabeshima T. The allosteric potentiation of nicotinic acetylcholine receptors by galantamine ameliorates the cognitive dysfunction in beta amyloid 25-35 i.e.v.-injected mice: Involvement of dopaminergic systems. Neuropsychopharmacology. 2007; 32:1261-1271.
Zeinali et al. Sources of marine superoxide dismutases: Characteristics and applications. International Journal of Biological Macromolecules 79 (2015) 627-637.
Zhou and Prognon. P. Raw material enzymatic activity determination. A specific case for validation and comparison of analytical methods—the example of superoxide dismutase (SOD). J. Pharm. Biomed. Anal. 2006, 40, 1143-1148.

1-13. (canceled)

14. A product containing a vegetal active extract containing SOD and a fenugreek extract.

15. The product of claim 14, wherein the vegetal active extract containing SODs is a melon juice concentrate containing SOD.

16. The product of claim 15, wherein the melon juice concentrate containing SOD is a coated freeze-dried melon juice concentrate containing a level of SOD ranging from 5 to 25 SOD U/mg powder.

17. The product of claim 16, wherein the melon juice concentrate containing SOD also contains carotenoids, vitamins and inorganic elements.

18. The product of claim 14, wherein the fenugreek extract is obtained by the following method:
a) The fenugreek seeds are extracted with supercritical CO₂ extraction, and further delipidated, and
b) The delipidated fenugreek seeds are further extracted by an alcoholic extraction.

19. The product of claim 18, wherein the fenugreek extract contains 4OH isoleucine and trigonelline.

20. The product of claim 19, wherein the fenugreek extract also contains homo-orientine and/or vitexine

21. The product of claim 14, wherein vegetal active extract containing SOD and fenugreek extract are in the same composition or in separate compositions of a kit.

22. The product claim 14, wherein it is intended for administration via oral, nasal or parenteral route.

23. The product of claim 14, wherein it is intended for oral administration.

24. The product claim 14, that is a nutritional product with a food-grade excipient.

25. The product of claim 24, that is a nutritional product with a food-grade excipient in the form of a tablet, hard capsule, soft capsule, effervescent tablet, sachet or stick to be diluted, chewing gum, beverages, juices, yoghurt, confectionery, biscuit or bars.

26. A method for improving cognitive function, or one condition selected from perception, memory, attention, and/or reasoning in a subject in need thereof, comprising the administration of a nutritional product of claim 24.

27. The method of claim 26, for use in the treatment or prevention of a condition selected from the group consisting of cognitive disorder, mood disorder, stress, and anxiety disorder.

28. The method of claim 26, wherein the nutritional product is intended for a daily administration.

29. The method of claim 28, wherein the nutritional product is intended for a daily administration in a dose equal or equivalent 1 to 8 IU SOD and 2,8 to 22.8 mg Fenugreek extract per kilogram (kg) body weight.

30. The product according to claim 14, that is a pharmaceutical product with a pharmaceutical excipient.

31. A method for the treatment or prevention of neurodegenerative disease in a patient in need thereof, comprising the administration of a pharmaceutical product of claim 30.

32. The method of claim 31, wherein the neurodegenerative disease is selected from the group consisting of Mild Cognitive Impairment (MCI), Alzheimer's disease and Parkinson's disease in a patient in need thereof.
